(19) United States
(12) Patent Application Publication
(10) Pub. No.: US 2009/0012590 A1
(43) Pub. Date: Jan. 8, 2009

(54) Neural Conductor

(75) Inventors: Michael Inman, Gainsville, FL (US); David Thompson, Houston, TX (US)

Correspondence Address: CONLEY ROSE, P.C.;David A. Rose, P. O. BOX 3267, HOUSTON, TX, 77253-3267, US

(73) Assignee: CYBERONICS, INC., Houston, TX (US)

(21) Appl. No.: 11/773,317

(22) Filed: Jul. 3, 2007

(51) Int. Cl. A61N 1/02

(52) U.S. Cl. 607/115

(57) ABSTRACT

Methods and devices for reducing current spread in nerve stimulation therapy are described herein. The disclosed methods and devices utilize a novel neural conductor placed between electrodes in an implantable medical device. The neural conductor provides a path for the current to pass through rather than allowing current to spread to neighboring tissues. In an embodiment, an electrode assembly for reducing current spread in nerve stimulation therapy comprises a first and a second electrode adapted to be coupled to a target nerve. In addition, the electrode assembly comprises a neural conductor which is spaced apart from the first and second electrodes.

### CROSS-REFERENCE TO RELATED APPLICATIONS

Not applicable.

### BACKGROUND

1. Technical Field

This invention relates generally to the field of implantable medical devices. More specifically, the invention relates to a neural conductor for reducing current spread from electrodes into surrounding tissue during nerve stimulation.

2. Background

Various diseases and disorders of the nervous system are associated with abnormal neural discharge patterns. One treatment regimen for such diseases and disorders includes drug therapy. Another treatment technique includes implanting in the patient's body a medical device having a pulse generator for electrically stimulating a target location of the patient's neural tissue. In one such available treatment for epilepsy, an electrical signal is applied to the vagus nerve by a neurostimulator device substantially as described in one or more of U.S. Pat. Nos. 4,702,254, 4,867,164, and 5,025,807, all of which are incorporated herein by reference.

A common problem in applying an electrical signal to nerve tissue is current spread. As used herein, current spread is the undesirable dispersion of the stimulus current into tissue surrounding the target tissue. It reduces the amount of electrical current flowing through the target nerve tissue, and therefore constitutes, at best, wasted energy. Additionally, however, current spread may result in an otherwise therapeutically effective electrical signal failing to achieve efficacy. Furthermore, current spread may cause undesired, uncomfortable and potentially harmful physiological side effects such as muscle and/or nerve twitching, or voice modulation.

Others have attempted to compensate for the problem of current stimulus spread by simply increasing the intensity level of the electrical stimulus, whereby the neural response to stimulation occurs despite the current shunted through undesired paths. Increased stimulus current levels increase the possibility of tissue damage, however. In addition, the increased stimulus current may also spread through undesired paths in inactive tissue, reaching the active nerve tissue at a level sufficient to produce a false positive response or indication of stimulation, again reducing the efficacy and accuracy of the nerve stimulation.

Consequently, there is a need for a device to improve conduction of current from electrode to electrode in electrical nerve stimulation to reduce current spread.

### BRIEF SUMMARY

Methods and devices for reducing current spread in nerve stimulation therapy are described herein. The disclosed methods and devices utilize a novel neural conductor placed between electrodes in an implantable medical device. The neural conductor provides an electrical path for the current to pass through, thereby reducing or eliminating the spread of current to neighboring, non-target tissues. Further aspects and advantages of embodiments of the invention are described in more detail below.

In one embodiment, an electrode assembly for reducing current spread in nerve stimulation therapy comprises a first and a second electrode adapted to be coupled to a target nerve. In addition, the electrode assembly comprises a neural conductor spaced apart from the first and second electrodes. As such, the neural conductor is not in direct electrical contact with either of the first and second electrodes.

In another embodiment, an implantable medical device comprises a first and a second electrode adapted to be placed in contact with a target nerve, and a neural conductor disposed between the first and second electrodes. The neural conductor is spaced apart from the first and second electrodes such that the neural conductor is not in direct electrical contact with either of the first and second electrodes

In another embodiment, a method of reducing current spread in nerve stimulation therapy comprises coupling a first and a second electrode to a target nerve. The method also comprises placing a neural conductor between the first and second electrodes to reduce current spread. The neural conductor is spaced apart from the first and second electrodes so as not to be in direct electrical connection with either of said first and second electrodes. In addition, the method comprises applying an electrical signal to the target nerve via the first and second electrodes.

The foregoing has outlined rather broadly the features and technical advantages of the present invention in order that the detailed description of the invention that follows may be better understood. Additional features and advantages of the invention will be described hereinafter that form the subject of the claims of the invention. It should be appreciated by those skilled in the art that the conception and the specific embodiments disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### BRIEF DESCRIPTION OF THE DRAWINGS

For a detailed description of the preferred embodiments of the invention, reference will now be made to the accompanying drawings in which:

FIG. 1 illustrates an embodiment of an implantable medical device to provide an electrical signal to a target neural tissue;

FIG. 2 illustrates a close-up of electrodes and leads in an implantable medical device to provide an electrical signal to a target neural tissue;

FIG. 3 illustrates an embodiment of an electrode assembly with a neural conductor placed within a nerve;

FIG. 4 illustrates another embodiment of an electrode assembly with a neural conductor disposed on the external surface of the nerve;

FIG. 5 illustrates another embodiment of an electrode assembly with a plurality of neural conductors disposed on the external surface of the nerve;

FIGS. 6A-C illustrate various embodiments of an electrode assembly in which a neural conductor surrounds the external surface of the nerve

FIG. 7 illustrates another embodiment of an electrode assembly with a neural conductor; and

FIG. 8 illustrates another embodiment of an electrode assembly with a neural conductor disposed on the external surface of the nerve.

### NOTATION AND NOMENCLATURE

Certain terms are used throughout the following description and claims to refer to particular system components. This document does not intend to distinguish between components that differ in name but not function.

In the following discussion and in the claims, the terms “including” and “comprising” are used in an open-ended fashion, and thus should be interpreted to mean “including, but not limited to . . . ”. Also, the term “couple” or “couples” is intended to mean either an indirect or direct electrical connection. Thus, if a first device couples to a second device, that connection may be through a direct electrical connection, or through an indirect electrical connection via other devices and connections.

### DETAILED DESCRIPTION OF THE EMBODIMENTS

The following description is presented largely in terms of an exemplary type of electrical neurostimulation, i.e., vagus nerve stimulation (“VNS”). However, the disclosure and claims that follow are not limited to VNS, and may be applied to the delivery of any electrical signal to modulate the electrical activity of other cranial nerves such as the trigeminal and/or glossopharyngeal nerves, or to other neural tissue such as one or more brain structures of the patient, spinal nerves, and other spinal structures. Further still, other embodiments of the invention can be implemented to stimulate tissue other than nerves or neural tissue, such as cardiac tissue or other muscle tissue.

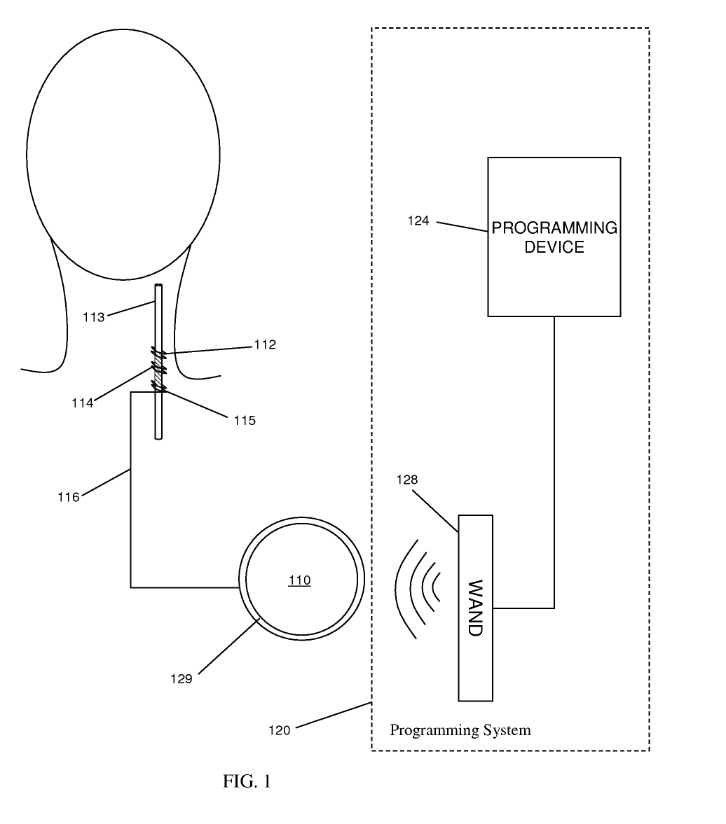

FIG. 1 illustrates an implantable medical device (“IMD”) 110 implanted in a patient. The IMD 110 may be representative of any of a variety of medical devices for providing electrical signal therapy to a target tissue. At least one preferred embodiment of the IMD 110 comprises a neurostimulator for applying an electrical signal to a neural structure in a patient, particularly a cranial nerve such as a vagus nerve 113. As used herein “stimulate” and “modulate” both refer to the delivery of such an electrical signal to a target body structure, regardless of whether the signal causes a particular effect such as an induction of an action potential in a stimulated nerve.

Referring still to FIG. 1, a lead assembly comprising one or more leads 116 is coupled to the IMD 110 and includes one or more electrodes, such as electrodes 112 and 114. Each lead 116 has a proximal end that connects to the IMD 110 and a distal end on which one or more electrodes are provided. The outer housing (or “can”) 129 of the IMD 110 preferably is electrically conductive and thus may also function as an electrode. The electrodes, such as electrodes 112, 114 and can 129, can be used to stimulate and/or sense the electrical activity of the target tissue (e.g., the vagus nerve 113). Strain relief tether 115 may also be included as an attachment mechanism that couples the lead assembly 116 to the nerve 113 to provide strain relief. An example of a suitable strain relief tether is described in U.S. Pat. No. 4,979,511, incorporated herein by reference.

Referring still to FIG. 1, external programming system 120 comprises a programming device 124. In the embodiment of FIG. 1, programming device 124 is coupled to a programming wand 128, which communicates with IMD 110 when placed on the patient's skin overlying the IMD. In systems where the external programming system 120 uses one or more channels in the Medical Implant Communications Service (MICS) bandwidths, the programming wand 128 may be omitted to permit more convenient communication directly between the programming device 124 and the IMD 110. The programming device 124 may comprise a personal computer, personal digital assistant (PDA) device, or other suitable computing devices consistent with the description contained herein. As explained below, the IMD 110 includes a transceiver (such as a coil) and the wand 128 also includes a transceiver. The transceivers in the IMD 110 and wand 128 permit signals to be communicated wirelessly and non-invasively between these components of the system. The programming system 120 provides one or more parameters to the IMD 110 to define the electrical signal to be applied to the target tissue.

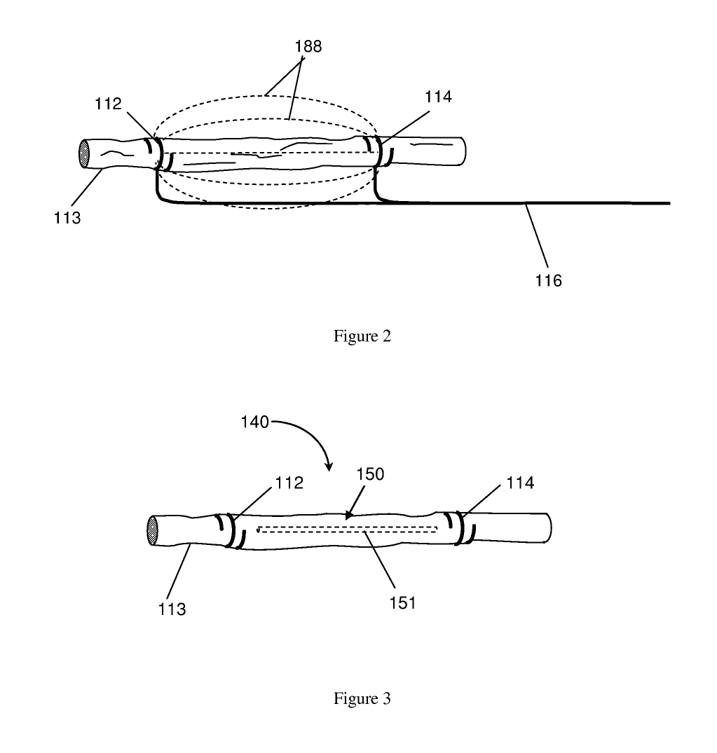

FIG. 2 is a close-up depiction of first and second electrodes 112, 114 and lead 116 coupled to target nerve 113. To simplify discussion of the present invention, strain relief tether 115 is not shown in the embodiments depicted in FIGS. 2-8. However, it will be understood that a strain relief tether could be incorporated into embodiments of the present invention without departing from its scope. Electric field lines 188 are depicted in FIG. 2 to show potential current spread away from target tissue 113 and into non-target tissue. As shown in FIG. 2, electrodes 112, 114 may be helical or spiral electrodes or other electrodes known in the art. See U.S. Pat. Nos. 6,600,956, 5,531,778, 5,351,394, 5,251,634, and 5,215,089, herein incorporated by reference in their entireties for all purposes. In addition, electrodes 112, 114 may be spaced at any distance from each other. In some embodiments, electrodes 112, 114 may be spaced at a distance ranging from 0.1 cm to about 10 cm, alternatively from about 0.5 cm to about 5 cm, and alternatively from about 1 cm to about 3 cm.

Referring now to FIG. 3, an embodiment of electrode assembly 140 comprises a neural conductor 150 for reducing current spread into non-target tissue. The neural conductor 150 may comprise one or more conductive elements 151. One or more conductive elements 151 are preferably disposed between the first and second electrodes 112, 114. Furthermore, one or more conductive elements 151 may be spaced apart from electrodes 112, 114, i.e., the conductive elements 151 are located so as not to contact electrodes 112, 114 to avoid the conductive element becoming an electrical shunt between electrodes 112, 114. However, in some embodiments, it is contemplated that conductive elements 151 may be in contact with one of the electrodes 112, 114, but not the other electrode as long as no direct electrical connection is formed between the electrodes 112, 114. For example, conductive elements 151 may be in contact with electrode 112, but spaced apart from electrode 114 and vice versa.

Conductive elements 151 may be spaced any appropriate distance from electrodes 112, 114. Neural conductor 150 does not serve as a direct connection or conduit for electricity in the traditional sense. Instead, the purpose of neural conductor 150 is to focus or localize the electric current passing from first electrode 112 to second electrode 114 to the target tissue (e.g., nerve 113) and reduce or eliminate undesirable current spread to non-target tissues. In one embodiment, the neural conductor 150 comprises a first end and a second end. The neural conductor may be positioned such that the first end is located near first electrode 112 and the second end is located near second electrode 114.

Without being limited to theory, electricity preferentially follows the path of least resistance. When traveling a long distance through a resistive volume conductor (e.g. a nerve), current tends to seek out the lowest impedance path. The neural conductor 150, therefore, provides a path of least resistance for the current passing from a first electrode (e.g., electrode 112) to second electrode (e.g., electrode 114). As such, neural conductor 150 may provide optimal recruitment of nerve fibers for more efficient stimulation of nerve 113 without loss of energy due to current spread.

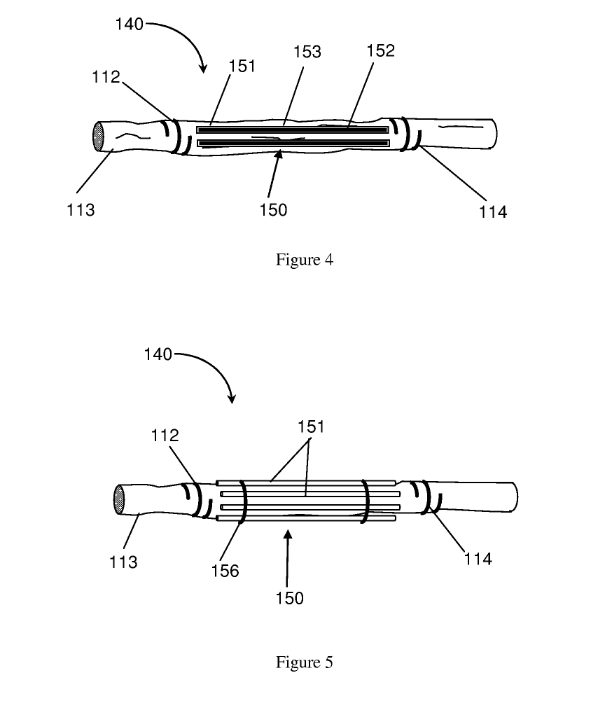

FIGS. 3-7 illustrate various embodiments of neural conductor 150. In FIG. 3, a conductive element 151 is inserted or disposed coaxially within the target nerve 113. However, one or more conductive elements 151, which make up neural conductor 150, may also be placed on the outer surface of the nerve 113 as shown in FIG. 4. In an embodiment, neural conductor 150 may have a plurality of conductive elements 151. The plurality of conductive elements 151 may be arranged circumferentially around the nerve 113 as seen in FIG. 4. Generally, conductive elements 151 are disposed parallel to nerve 113. However, it is contemplated that conductive elements 151 may also be placed in any orientation with respect to nerve 113 (e.g. perpendicular, angled, helically, etc.).

The one or more conductive elements 151 may comprise any materials capable of conducting electricity. In particular, conductive elements 151 may comprise a metal such as without limitation, iridium, copper, silver, gold, platinum, aluminum, nickel, steel, metal alloys, or combinations thereof. Additionally, the conductive elements may preferably be selected to provide biocompatibility. Moreover, conductive elements 151 may comprise a non-metallic material such as without limitation, a conductive polymer or carbon.

Conductive elements 151 may comprise any suitable geometry. For example, in FIG. 5, conductive elements 151 comprise a cylindrical, a rectangular or tubular shape. The conductive elements 151 may be hollow or solid (not hollow). In an embodiment, conductive elements 151 may be held in place by tie-downs or straps 156 as shown in FIG. 5. Alternatively, conductive elements 151 may be attached to nerve 113 through sutures or a tissue adhesive.

In another embodiment, conductive elements 151 may each comprise a strip 152 disposed on a thin substrate layer 153 as shown in FIG. 4. Strip 152 preferably comprises a thin metal layer. In an embodiment, the thin metal layer may have a thickness ranging from about 1/5,000 inch to about 1/10 inch, alternatively from about 1/1,000 inch to about 1/50 inch, alternatively from about 1/500 inch to 1/100 about inch. Strip 152 may be attached to substrate layer 153 through any means such as without limitation, adhesive, lamination, printing, lithography and the like. The substrate layer 153 may be at least partially coated with an adhesive so as to couple the substrate layer, and thus the conductive element 151, to the nerve 113. Substrate layer 153 may comprise any suitable biocompatible material such as without limitation, silicone, polyurethane, nylon, polyester, polytetrafluoroethylene, polyamide, combinations thereof, and the like.

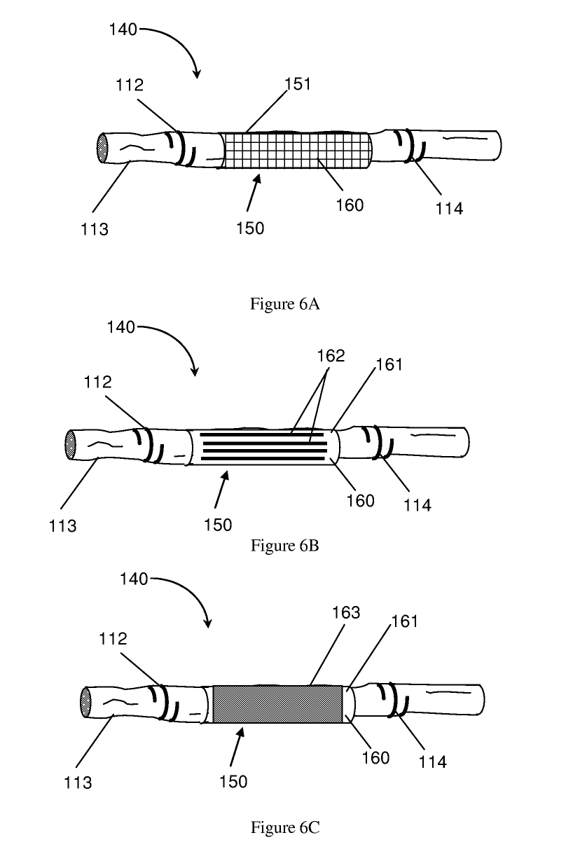

As shown in FIG. 6A, other embodiments of the neural conductor 150 may include a conductive element 151 which comprises a conductive sleeve or cuff 160. Cuff 160 may comprise a continuous mesh or fabric of conductive material or alternatively, may comprise a continuous film of conductive material. In other embodiments, cuff 160 may only partially surround the nerve. That is, cuff 160 may have a semi-circular or a C-shaped cross-section. The conductive material may be any suitable metal or conductive non-metal such as described above. Generally, cuff 160 may comprise a metal mesh made of conductive metal fibers. In a particular embodiment, cuff 160 may comprise carbon fiber.

In the embodiment of FIG. 6B, cuff 160 may comprise a substrate layer 161 surrounding the nerve 113 having one or more strips 162 of conductive material attached to substrate layer 161. Strips 162 may be attached to inner or outer surface of substrate layer 161. Strips 162 may be disposed parallel to nerve 113, as shown in FIG. 6B, or may be disposed perpendicular to nerve 113, at predetermined angles to the nerve, or another desired position relative to the target tissue. Alternatively, in FIG. 6C, cuff 161 may comprise a substrate layer 161 with a continuous sheet or layer 163 of conductive material attached to substrate layer 161. Continuous layer 163 may also be disposed on inner or outer surface of substrate layer 161.

Substrate layer 161 preferably is a biocompatible flexible material such that cuff 160 may conform and move with the nerve 113. Cuff 160 may be attached to nerve 113 through any suitable means. For example, in an embodiment, cuff 160 may be sutured, stapled, or glued together.

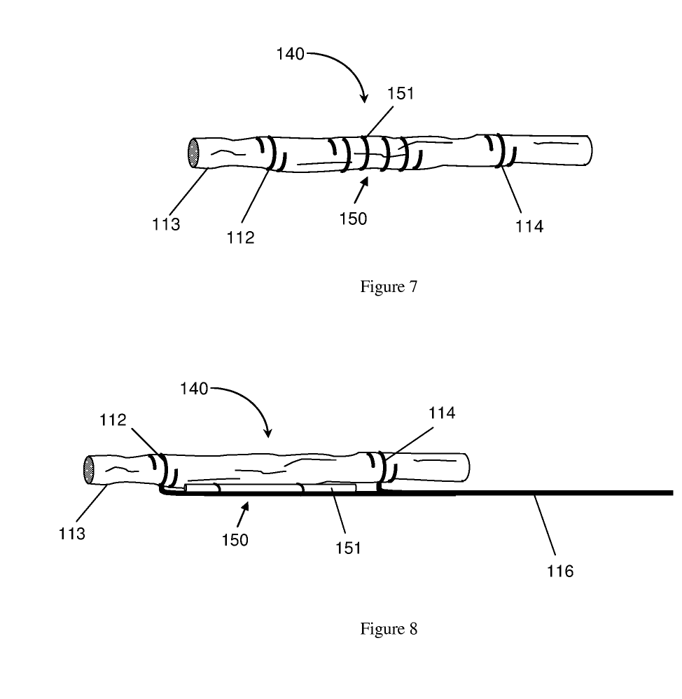

In the embodiment of FIG. 7, conductive element 151 may comprise a helical or spiral geometry as shown in FIG. 4. Helical or spiral conductive element 151 may wrap around the target tissue, such as nerve 113, to hold itself in place. Although only one helical conductive element 151 is depicted in FIG. 4, a plurality of helical conductive elements 151 may be placed between first and second electrodes 112, 114 for further reduction of current spread. Helical conductive element 151 may be placed on the nerve by a surgeon by gently bending and wrapping the conductive element 151 around nerve 113 or other target tissue.

In an embodiment, neural conductor 150 may be held in place by lead 116. Neural conductor 150 may be coupled to lead 116 through any suitable means such as without limitation, straps, tie-downs, adhesive, sutures, etc. Moreover, conductive element 151 may comprise any of the structure elements in the embodiments described above (e.g. strips, tubes, wires, and the like).

While embodiments of the invention have been shown and described, modifications thereof can be made by one skilled in the art without departing from the spirit and teachings of the invention. The embodiments described and the examples provided herein are exemplary only, and are not intended to be limiting. Many variations and modifications of the invention disclosed herein are possible and are within the scope of the invention. Accordingly, the scope of protection is not limited by the description set out above, but is only limited by the claims which follow, that scope including all equivalents of the subject matter of the claims.

The discussion of any reference in the Background is not an admission that such references are prior art to the subject matter of this disclosure, especially any reference that may have a publication date after the priority date of this application. The disclosures of all patents, patent applications, and publications cited herein are hereby incorporated herein by reference in their entirety, to the extent that they provide exemplary, procedural, or other details supplementary to those set forth herein.

What is claimed is:

1. An electrode assembly for reducing current spread in nerve stimulation therapy, said electrode assembly comprising:
a first electrode and a second electrode each adapted to be coupled to a target nerve; and
a neural conductor disposed between said first and second electrodes, wherein said neural conductor is spaced apart from said first and second electrodes so as not be in direct electrical connection with either of said first and second electrodes.

2. The electrode assembly of claim 1 wherein said neural conductor is adapted to be disposed within the target nerve.

3. The electrode assembly of claim 1 wherein said neural conductor is adapted to be disposed on the outer surface of the target nerve.

4. The electrode assembly of claim 1 wherein said neural conductor comprises one or more conductive elements.

5. The electrode assembly of claim 4 wherein said one or more conductive elements comprises a strip, a wire, a tube, a cuff, or combinations thereof.

6. The electrode assembly of claim 5 wherein said one or more conductive elements comprises a cuff having a semi-circular cross-section.

7. The electrode assembly of claim 5 wherein said one or more conductive elements comprises a cuff comprising a mesh, a metal film, or a fabric.

8. The electrode assembly of claim 1 wherein said neural conductor comprises a metal, carbon, a conductive polymer, or combinations thereof.

9. The electrode assembly of claim 1 wherein said neural conductor is disposed on a substrate layer.

10. The electrode assembly of claim 9 wherein said substrate layer forms a cuff.

11. The electrode assembly of claim 9 wherein said substrate layer comprises a polymer.

12. The electrode assembly of claim 1 wherein said neural conductor comprises a first end and a second end, and wherein said first end is positioned near said first electrode and said second end is positioned near said second electrode.

13. The electrode assembly of claim 1 wherein said neural conductor is helical.

14. The electrode assembly of claim 1 wherein the first and second electrodes are coupled to a lead, and wherein said neural conductor is attached to the lead.

15. An implantable medical device comprising:
first and second electrodes adapted to be placed on a target nerve;
a signal generator coupled to said first and second electrode; and
a neural conductor disposed between said first and second electrodes, wherein said neural conductor is spaced apart from said first and second electrode so as not to be in direct electrical connection with either of said first and second electrodes.

16. The implantable medical device of claim 15 wherein said neural conductor is adapted to be implanted coaxially within the target nerve.

17. The implantable medical device of claim 15 wherein said neural conductor is adapted to be placed on the outer surface of the target nerve.

18. The implantable medical device of claim 15 wherein said neural conductor comprises one or more conductive elements.

19. The implantable medical device of claim 15 wherein said one or more conductive elements comprises a strip, a wire, a tube, a cuff, a spiral, or combinations thereof.

20. The implantable medical device of claim 15 further comprising a lead coupling said first and second electrodes to said signal generator, and wherein said neural conductor is attached to said lead.

21. A method of reducing current spread in nerve stimulation therapy, the method comprising:
coupling a first and second electrode to a target nerve; and
placing a neural conductor between the first and second electrodes to reduce current spread, wherein the neural conductor is spaced apart from the first and second electrodes so as not to be in direct electrical connection with either of the first and second electrodes; and
applying an electrical signal to the target nerve via the first and second electrodes.

22. The method of claim 21 wherein placing the neural conductor comprises inserting the neural conductor coaxially into the target nerve.

23. The method of claim 21 wherein placing the neural conductor comprises placing the neural conductor on the outer surface of the target nerve.

24. The method of claim 21 wherein the target nerve is the vagus nerve.
